(19) United States
(12) Patent Application Publication
(10) Pub. No.: US 2014/0234394 A1
(43) Pub. Date: Aug. 21, 2014

(54) BENEFICAL CONTACT SURFACES FOR FUNCTIONAL OBJECTS

(71) Applicants: William A. McDonough, Charlottesville, VA (US); Aly Khalifa, Raleigh, NC (US)

(72) Inventors: William A. McDonough, Charlottesville, VA (US); Aly Khalifa, Raleigh, NC (US)

(21) Appl. No.: 13/770,184

(22) Filed: Feb. 19, 2013

(51) Int. Cl. A61K 9/00; A61K 31/315; A61K 33/08

(52) U.S. Cl. CPC A61K 9/0014; A61K 33/08; A61K 31/315
USPC 424/444; 424/692; 514/494

(57) ABSTRACT

An enhanced article which has a beneficial contact surface intended to allow bioabsorption of beneficial materials through the user's contact with the enhanced article in use. A functional or ornamental article which has at least one contact surface intended to come in contact with a human user includes a beneficial contact surface for covering the article's contact surface and providing a beneficial surface contact compound adapted to come in contact with a human user touching the article. The beneficial contact surface is connected to the article so that the contact surface of the article is covered by the beneficial contact surface. The presence of the beneficial surface contact compound in the beneficial contact surface means results in the exposure of the user's skin to the beneficial surface contact compound to allow absorption by the user. The article can be any functional or ornamental object which in its regular use comes in contact with a user's skin, including but not limited to such objects as a cellphone, doorknob, packaging, clothing, brushes, laptops and shower curtains. Also, a process by which articles can be converted into enhanced articles through addition of a beneficial contact surface which provides bioabsorption of beneficial materials added as contact surfaces for the articles through various techniques.

This application claims the priority of U.S. Provisional Patent Application Ser. No. 61/599,710, filed on Feb. 16, 2012.

### BACKGROUND OF THE INVENTION

The invention relates generally to functional products that have beneficial contact surface compounds either integrated into the functional product or added to the original surface with surface compounds that are transferable through contact and, more particularly to objects and articles having surface characteristics that include beneficial contact surface compounds for the human body that are bioabsorbable through skin contact with the surfaces of the product, objects and articles.

In the past, functional articles and objects which come in contact with the human body during their normal use have been optimized for purposes of the functional article. For example, the elements of the door handle are optimized for the functional operation of the door handle, ease in gripping and manipulating the handle for those with limited flexibility and other handicaps, but no focus on the effect of human body contact with the materials and surfaces of the door handle. Apart from design goals to avoid sharp surfaces which might injure a user or use of known carcinogens, there has been very limited focus on the surface contact materials.

In the past there have been various transdermal patches to deliver medications and nutrients of various sorts through the skin of the human body for various purposes. Some of these are to apply medication over a period of time, others to provide medical treatments such as nicotine supplements to allow cigarette smokers to stop smoking and systems which provide continuous pain relief through the transdermal patches. These systems are intended to effect the health and well being of human beings by actively adding beneficial materials through the skin of the human being.

However, there is a need for materials which are placed as beneficial contact surfaces on functional articles which, through their regular use, come in contact with the human body and which have traditionally been designed with surfaces made of materials which have become known to produce negative interactions with the human body. The need is not intended to transfer the quantities of beneficial compounds necessary for therapeutic levels, but still provide a positive effect.

For example, some computer laptops have been designed with aluminum surfaces, incorporated primarily because of the light weight nature of aluminum and the surface finish creating a positive, sleek and stylish appearance. However, recent research has noted a possible correlation between the presence of aluminum and the onset of Alzheimer's disease. While there is conflicting research at this point, the presence of aluminum surfaces on functional items such as laptop computers, which are frequently touched, handled and held by human beings during their use raises the specter of possible negative consequences to the interaction on a regular basis with an aluminum surface. Accordingly, there is a need, not only for a protective layer preventing contact with the aluminum surface, but also with a contact surface having beneficial interactive qualities with the human body.

Similarly, certain surfaces which have the potential for negative interaction with the human body are standard paper, plastic or board packaging with traditional inks which include a variety of toxic and potentially toxic materials, including heavy metals and the like known to be generally bad for humans if absorbed. Thus, there is a need for a beneficial contact surface for packaging in which inks based on beneficial minerals and elements are utilized to prevent negative interaction with and enhance positive or beneficial interaction with the human body.

In addition, there are a wide variety of surface materials which are utilized currently which provide no beneficial engagement with the human body and provide potentially toxic elements and compounds into contact with the human body, either as a result of design considerations related to the functional article or treatment processes utilized which retain or develop potentially toxic materials as an outgrowth of the manufacturing or finishing processes. Accordingly, there is a need for materials which can be added, either as part of the manufacturing process, as part of a surface applied during the process such as a paint or lacquer or, subsequent to production, through various processes which apply a surface of materials incorporating beneficial components.

### SUMMARY OF THE INVENTION

The invention is generally directed to beneficial contact surfaces for functional articles, either integral with, applied to or added to existing surfaces to provide beneficial interaction with the human body and to prevent contact with less beneficial contact surfaces and surface materials.

Accordingly, an object of the invention is to provide an improved surface application system whereby beneficial materials are integrated, applied to or added to the surface of functional articles which come into contact with the human body to provide enhanced interactivity with the human body and reduce exposure to potentially unsafe surface materials.

Another object of the invention is to provide through a variety of techniques, the addition of a beneficial contact surface for articles which come in contact with humans during their regular use, through introduction of beneficial minerals, compounds or materials integrated as surface layers, embedded into liquids intended to dry into surface layers like paints or lacquers, formed with substrates affixed to the article, embedded as nanofibers into fibrous or woven materials and as embedded nanofibers and similar forms within carriers. Nanofibers are typically 1000 nanometers in diameter or less.

The invention accordingly comprises the features of construction, combinations of elements and arrangements of part and processes which will be exemplified in the constructions and processes as hereinafter set forth, and the scope of the invention will be indicated in the claims.

### BRIEF DESCRIPTION OF THE DRAWINGS

The present invention will be described in more complete detail with frequent reference being made to the Figures identified below.

FIG. 1 is a diagrammatic view of the outer layers of the skin of a human body;

FIG. 2 is a diagrammatic Fig. of possible uses of beneficial contact surfaces;

FIG. 3 is an elevational view of a functional article with a beneficial contact surface in accordance with a preferred embodiment of the invention;

FIG. 4 is a coating layer including a beneficial contact surface material in accordance with another preferred embodiment of the invention;

FIG. 5 is a perspective view of a portion of a beneficial contact surface constructed in accordance with another preferred embodiment of the invention;

FIG. 6 is a cutaway top plan view of another beneficial contact surface layer in accordance with another preferred embodiment of the invention;

FIG. 7 is a top plan view of a woven beneficial contact surface in accordance with another preferred embodiment of the invention;

FIG. 8 is a perspective view of a doorknob constructed with a beneficial contact surface in accordance with a preferred embodiment of the invention;

FIG. 9 is a perspective view of another doorknob with a beneficial contact surface constructed in accordance with another preferred embodiment of the invention;

FIG. 10 is a perspective view of a series of packages including beneficial contact surfaces in accordance with another preferred embodiment of the invention;

FIG. 11 is a side elevational view of a sock with beneficial contact surfaces constructed in accordance with another preferred embodiment of the invention;

FIG. 12 is a perspective view of gloves constructed with beneficial contact surfaces in accordance with another preferred embodiment of the invention;

FIG. 13 is a perspective view of a brush constructed with beneficial contact surfaces in accordance with another preferred embodiment of the invention;

FIG. 14A is a perspective view of a cell phone being used by a human being and the contact therebetween with a cell phone constructed with beneficial contact surfaces in accordance with another preferred embodiment of the invention;

FIG. 14B is a perspective view of the cell phone of FIG. 14A being held by the user's hand;

FIG. 14C is a front elevational view of the back surface of the cell phone of FIGS. 14A and 14B constructed with a beneficial contact surface in accordance with another preferred embodiment of the invention; and

FIG. 15 is a perspective view of a shower curtain constructed with beneficial contact surfaces in accordance with another preferred embodiment of the invention.

### DETAILED DESCRIPTION OF THE PREFERRED EMBODIMENTS

In our ever more complicated and high-technologically advanced environment the human body comes in contact with a great number of surfaces of functional articles during the course of the normal day. From the sheets and blankets when laying in bed, to the carpeting when one walks to the bathroom, to the surfaces in the bathroom, the articles used in the morning toilette routine, clothing worn during the day, door handles touched as one enters or leaves a room, and innumerable other objects in the workplace and other environments, we are constantly making contact with our environment.

Through technological advances many of the products and articles that we come in contact with are manufactured by processes which introduce materials either not found in our natural environment or added in a fashion which combines to create surface contact materials which are potentially hazardous or toxic to humans. Over time, we have become far more sensitive to the various materials in our environment with many people developing allergies to many materials which, in the past, were nontoxic or not as toxic. Much in the same way that various allergies such as peanut or other nut allergies have become far more prevalent or virulent than in the past, people are developing heightened sensitivities to environmental factors previously not considered to be either toxic or problematic. However, there has been a failure to adequately safeguard the human users of a wide variety of functional articles through design processes focused almost entirely on enhancing the functional aspects of the article without appropriate consideration of the human article interface.

As people have become more sensitive to the enhanced toxicity of the world in which we exist, there has developed the need to reduce and prevent the contact with surfaces either including, treated with or having added materials which have the potential for harming humans through contact.

For example, there has been a growing scientific awareness of the possibility that contact with aluminum surfaces may result in minute absorption of aluminum which has been tied to possible increase in a likelihood of developing Alzheimer's disease or other conditions. Similarly, many products which we come in contact with, including clothing, metal surfaced products and the like, contain trace amounts of materials which, in larger quantities, are known to be toxic. And with continued contact the low level toxicity becomes more likely to create negative interactions directly related to the product of dose and delivery time. Particularly with respect to those articles which are part of our normal existence and which we touch many times during the course of each day, such as the handle of a doorknob, brushes, clothing, carpet fibers, packaging and the like, there are enhanced possibilities of delivering toxic materials to our bodies through contact with our skin.

One particularly significant concern is with respect to cell phones which are both held and pressed against our cheeks and ears as they are used. Other examples include gloves or socks which rest against our skin and functional articles utilized at the workplace such as staplers, copying machines and even desk tops.

On the other hand, there can be a reformation of these articles through the development and application of surfaces including minerals, materials, compounds and the like which are known to have beneficial impacts on the human body. These beneficial contact surfaces can be applied in a variety of ways to promote beneficial integration of the functional articles with the human body contact, as well as protecting against the contact with the undesirable, potentially hazardous or toxic surface materials currently in use.

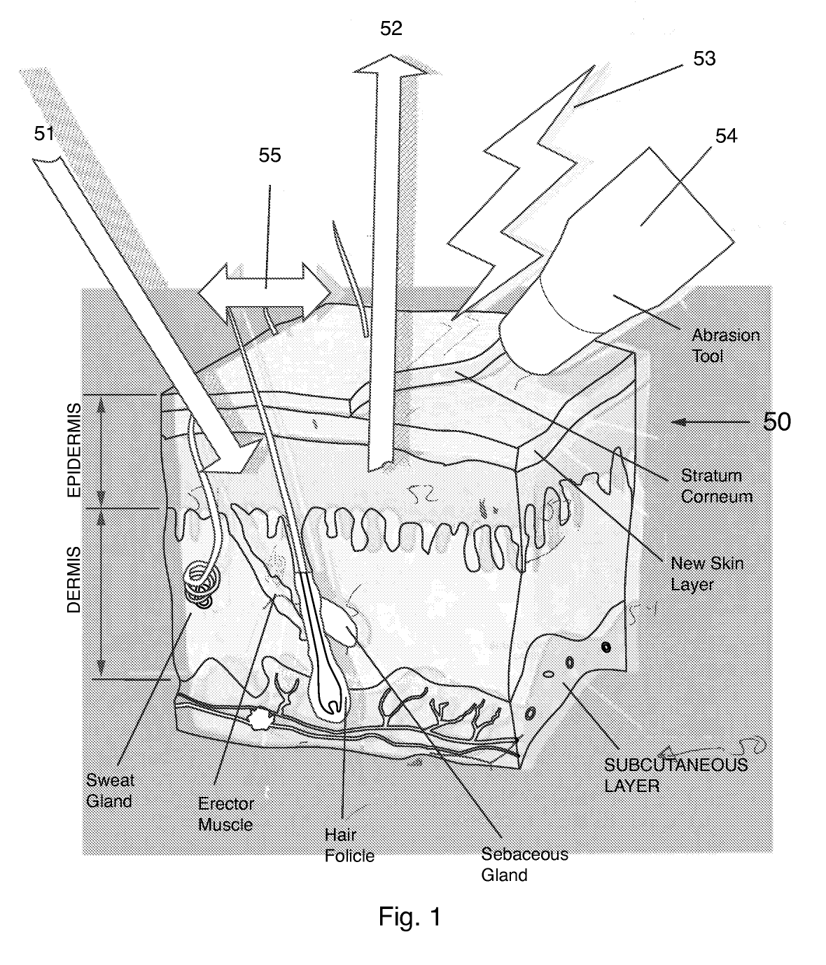

Reference is first made to FIG. 1, wherein a cutaway view of the human skin is shown. The skin 50 is shown with its various layers, including the stratum, corneum, new skin layer, and the remainder of the epidermis and the dermis including sweat glands, erector muscles, hair follicles, subcutaneous glands and a subcutaneous layer. Arrow 51 shows the manner in which the skin can receive materials through additive treatments. Arrow 55 shows treatments which can be protective or disinfecting removing potentially hazardous materials from the surface. Arrow 52 shows a pathway of removing toxins. Bolt 53 shows how adding energy to the surface of the skin 50 increases the transdermal effects. Abrading tool 54 shows how abrading the surface, similarly, can increase the transdermal effects and cause materials to be absorbed more effectively through the skin. Each of these pathways is important, both in terms of protecting against the passage of harmful materials across the skin boundaries and in assuring that desired or beneficial materials have an easier time being absorbed through the skin and providing the benefits of that contact.

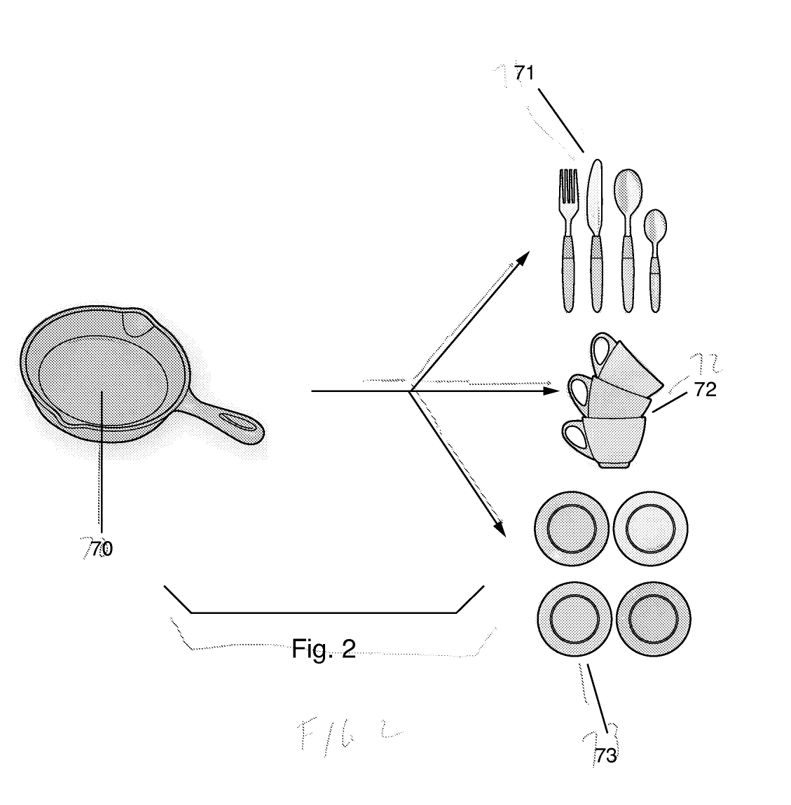

Reference is next made to FIG. 2 wherein a cast iron pan 70 is shown. It is known that there are generally positive associations with the iron from the cast iron pan being absorbed into the food cooked in it and thereby enhancing the nutritional value of that which is received. Similarly, it would be useful to provide beneficial contact surfaces to the cutlery 71, cups 72 and plates 73 which are used by us as we eat and drink and which regularly come in contact with our mucous membranes within the mouth which are far more receptive to the passage of materials, whether beneficial or hazardous. These types of functional articles are particularly well suited to having beneficial contact surfaces in accordance with preferred embodiments of the invention on them either as an integrated component of the construction or by a surface layer applied to the article.

Reference is next made to FIG. 3, which shows a side elevational view of an article 100, having a top surface 101 and a beneficial layer 102 on top of that. The surface 101 is preferably nonhazardous and nontoxic but, to the extent that it includes potentially hazardous, toxic or even cancerous materials or compounds, the protective layer 102, which is formed of materials which are either beneficial or beneficial combined with those which are protective or neutral in interacting with humans.

There are various materials which are known to provide positive interactions with humans, including a variety of minerals and compounds which include magnesium, such as magnesium oxide and other materials. Also, zinc compounds, if not paired with cadmium, which is considered potentially toxic, and which is often found paired with zinc as it is mined and produced, can have similar benefits. Other materials can be utilized in accordance with the invention to be created as beneficial contact surfaces either alone or in combination with other materials having salutary effects on human contact. There is further description of beneficial compounds below. The layer 102 may either be a separate piece which is adhered to layer 100 or may be a very thin layer put down by deposition or electro-chemical means, such as plating or a gas diffusion system.

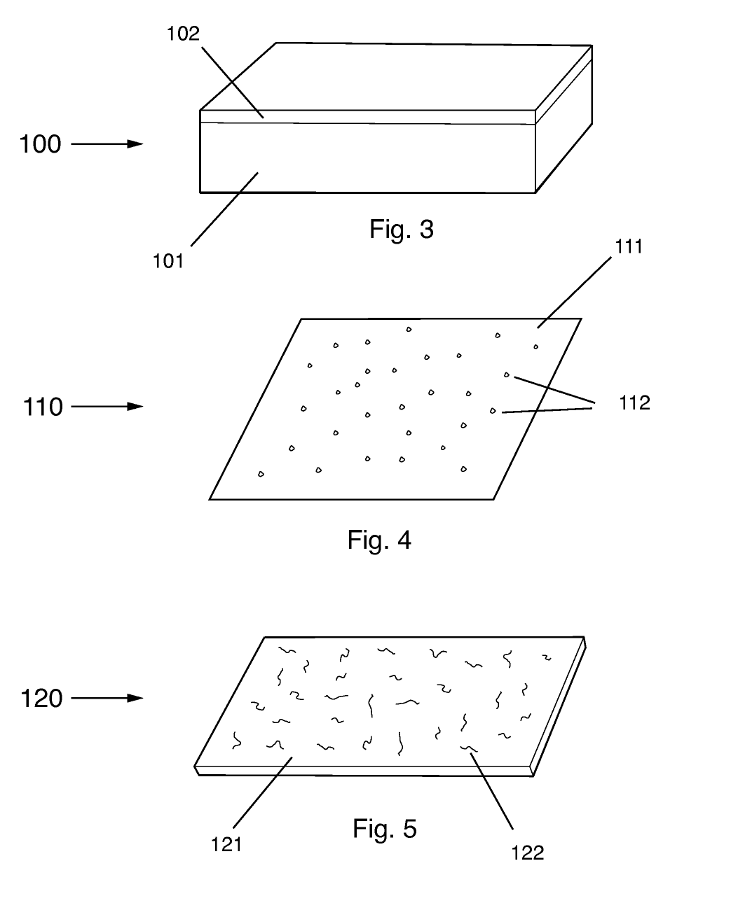

Reference is next made to FIG. 4 wherein a beneficial contact surface layer 110, constructed in accordance with another preferred embodiment of the invention, is depicted. Beneficial contact surface 100 includes a substrate 111 and beneficial materials 112 integrated into substrate 111. In preferred embodiments of the invention the substrate is formed of a material which is neutral to human contact and does not introduce any possible hazardous or toxic effects by contacting the human body. The presence of the beneficial material 112 can be adapted based upon the needs of the surface and to vary the degree of beneficial effect achieved through contact with surface 110. Generally, in this situation, surface 110 can be formed as a film or similar surface which can be applied to the surface of a designated functional article following the manufacturing of the functional article and prior to its final completion. In addition it can be formed of a material like latex or other moldable material which can be formed into a skin for a cellphone or similar covering to all or a portion of an article so that the contact surface with human interaction has the embedded beneficial contact compound in contact with the user's skin.

Reference is next made to FIG. 5 wherein a beneficial contact surface 120, constructed in accordance with another preferred embodiment of the invention, is depicted. Surface 121 includes a series of nano-fibers or similar small portions of the beneficial materials embedded in substrate 121. The nano-fibers, such as those of magnesium oxide, can be integrated into the substrate 121 in a variety of ways which allow for the beneficial material to interact with and be available for absorption through user's skin.

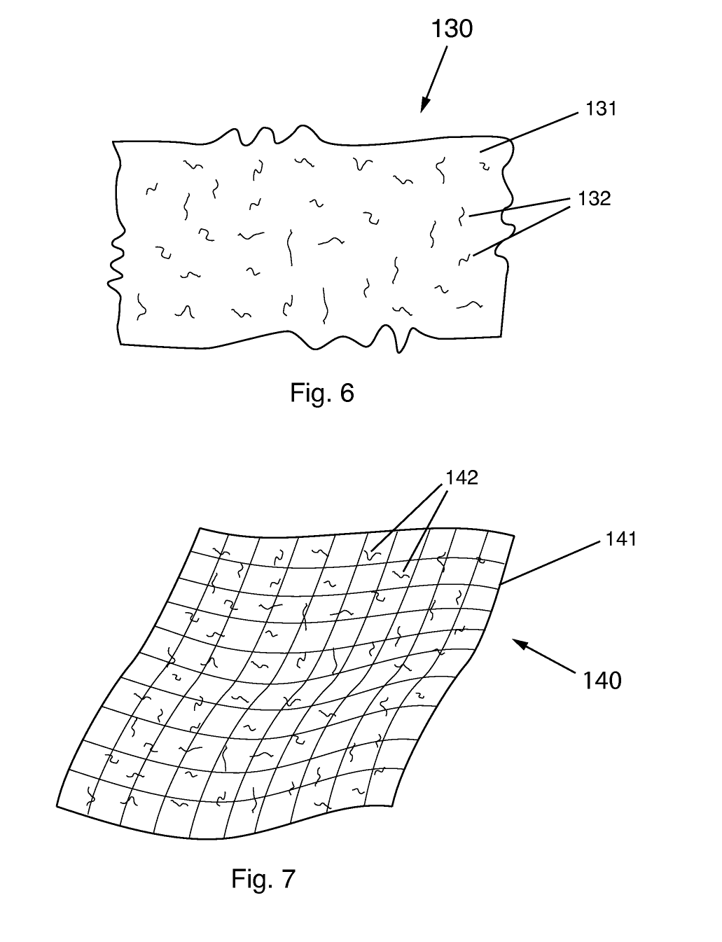

Reference is next made to FIG. 6 wherein a cutaway portion of an applicable surface layer 130, such as paint or stain, is applied to the functional article. The dried paint 131 is shown to include a series of nano-fibers or similar dissolved or included components of the beneficial material in the surface material 130. Generally, the fibers 132 would be mixed within the liquid paint or stain prior to its application and, as the paint dries on the surface, the nano-fibers 132 remain distributed throughout the surface of the dried paint 131, such that contact with the surface covered by the paint 131 would bring the user in contact with the nano-fibers of beneficial materials. Alternatively, the liquid form of the beneficial contact surface could be formed through use of a mold into a designated shape, which could then act like a skin for the article, like the latex or plastic cellphone covers.

Reference is next made to FIG. 7 wherein a stylized woven fabric or other material 140 is assumed. Fabric 140 includes woven fibers 141 in accordance with traditional formation of woven materials and includes nano-fibers 142, which are woven with the threads or other materials used to create the woven fabric. This approach is useful in connection with clothing, carpets, brushes or any materials in which the basic surface of the functional article is of a woven nature or made of fibers. The nano-fibers are integrated into the material to be woven and, as a result of their presence, are in a position where the human user would come into physical contact with many of the nano-fibers during the course of contact with the article.

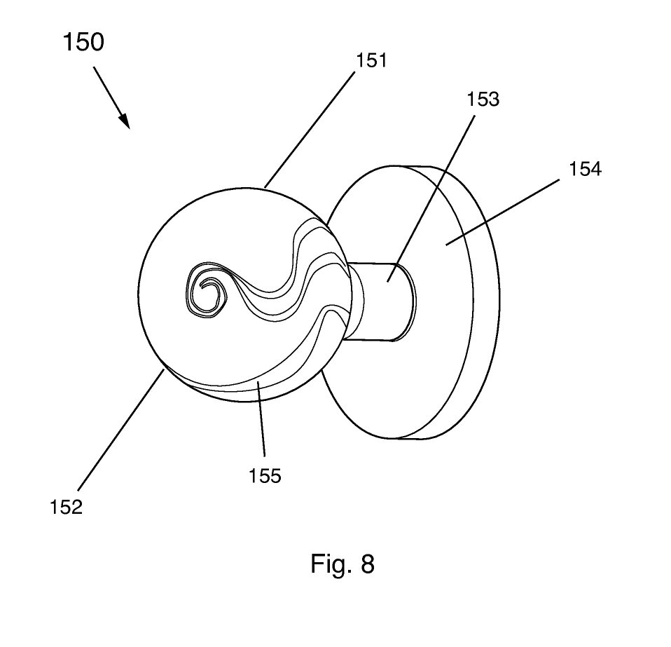

Reference is next made to FIG. 8 wherein a door handle constructed in accordance with a preferred embodiment of the invention is depicted. Doorknob 150 includes a film treatment on a substrate which allows the beneficial contact surface to be applied to many materials. This is similar to the approaches shown in FIG. 4, 5 or 6. The doorknob 150 includes color-coded multi-mineral zones 152 to increase whole-body health by providing access to a variety of different minerals or even vitamins. Doorknob 150 includes handle portion 155 connecting arm 153 and base 154.

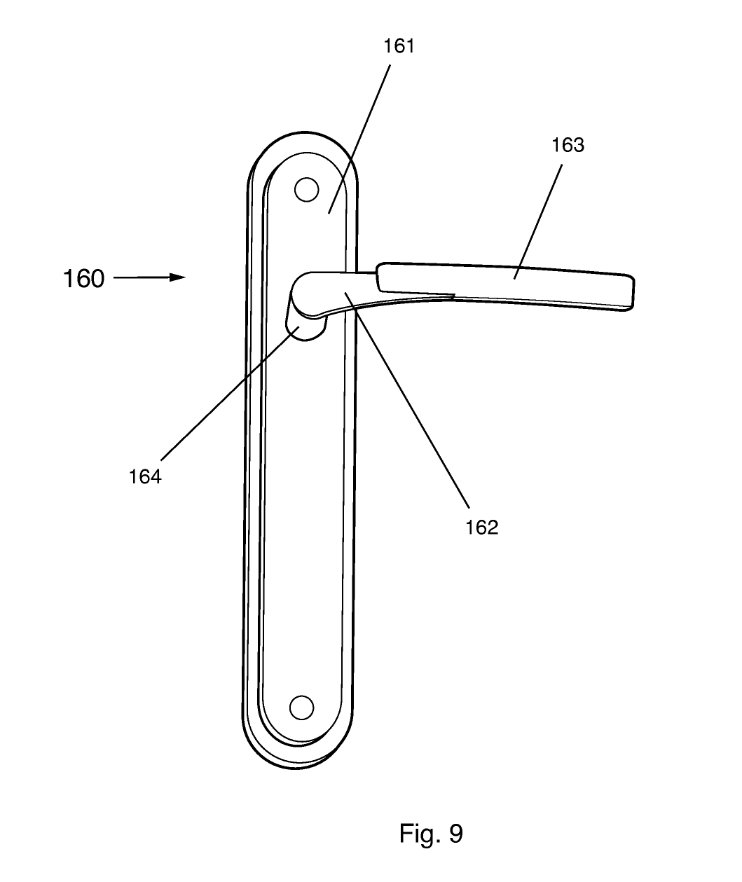

Reference is next made to FIG. 9 wherein a standard door handle construction 160 in accordance with another preferred embodiment of the invention is depicted. Door handle 160 includes mounting component 161, handle arm 162, beneficial contact surface 163 and standard mechanism 164. In this case, the beneficial contact surface 163 can be formed as a pure zinc or magnesium oxide component installed onto handle arm 162 in the way shown in FIG. 3, where the beneficial contact surface is a separate layer on top of the functional article. Here, the beneficial contact surface can be formed as an integral member secured through conventional means to the handle arm 162. Of course, the beneficial contact surface could also be formed by a beneficial compound or mineral embedded in a laminate either as in FIG. 4, 5 or 6 as well.

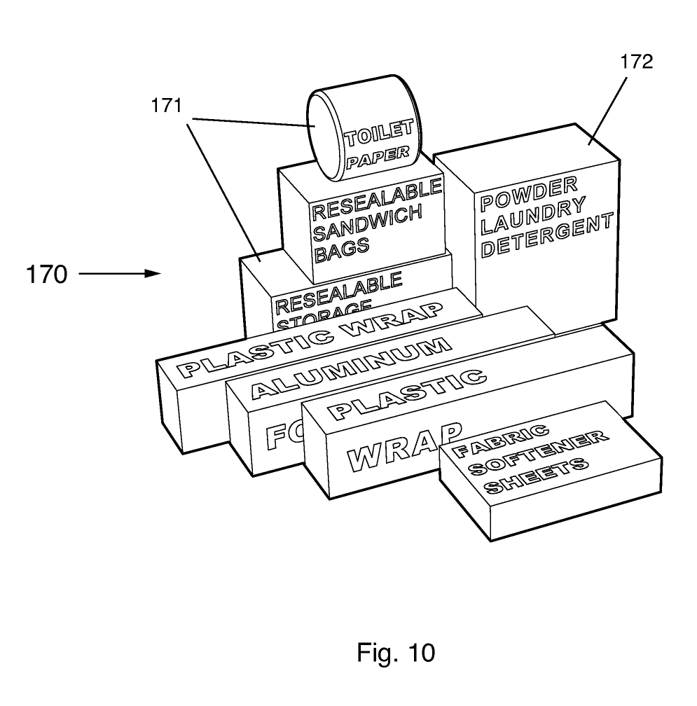

Reference is next made to FIG. 10 wherein packaging with beneficial contact surface is added through incorporating the beneficial materials into the ink used for the outside of the packaging. As shown in FIG. 10, packaging 170 includes standard paper, plastic or boards 171 following standard industry practice, with magnesium or zinc based inks 172 providing a beneficial contact surface of packaging so that, as the user interfaces with the packaging, he or she will make contact with the beneficial contact surface. This is most like the model of FIG. 6 where a liquid or powdery dye, such as shown in FIG. 6, is used to be applied to and to set on the surface of packaging 171. It could also be through application of a laminate embedded with the beneficial compound or other material to the outer surface of the conventional packaging.

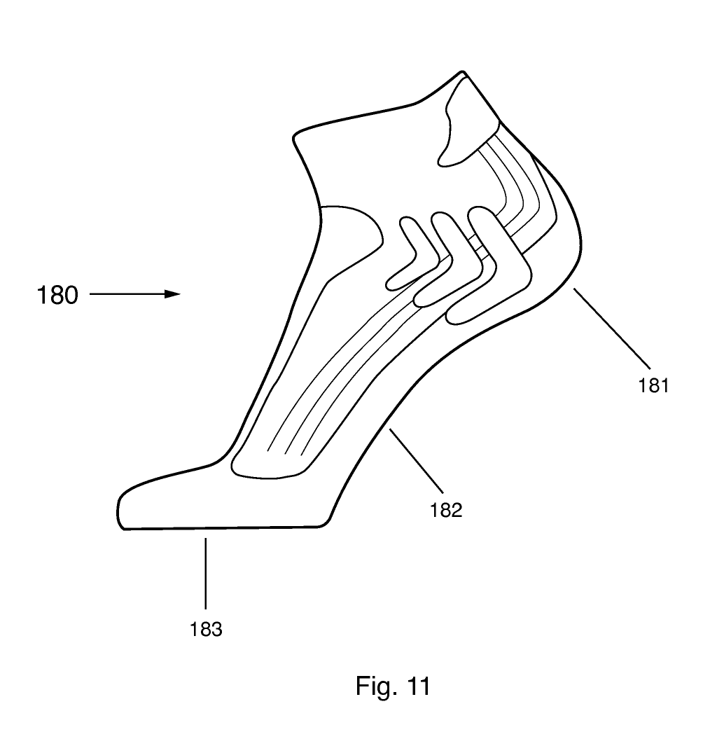

Reference is next made to FIG. 11 wherein a sock 180 constructed in accordance with another preferred embodiment of the invention is depicted. The sock includes abrasion inducing materials 183 at the toe region which can enhance the transdermal effects from beneficial materials implemented in the weave of the sock 180 through the use of nano-fibers or materials implemented onto the surfaces of the fibers used in forming the approaches shown in FIG. 7 and FIG. 4. Similarly, the osmotic pressure treatment may cause the beneficial contact surface materials to be implemented in region 182 where the user sweats through the sock, causing the sweat to allow the beneficial materials to be more readily absorbed into the user's foot. Next, in the heel region 181, where enhanced pressure may be present as the user stands, enhanced particle size of the beneficial materials either in connection with the embodiments of FIG. 4, 5 or 7, may be used to enhance the degree of absorption of the beneficial materials through the user's skin contact.

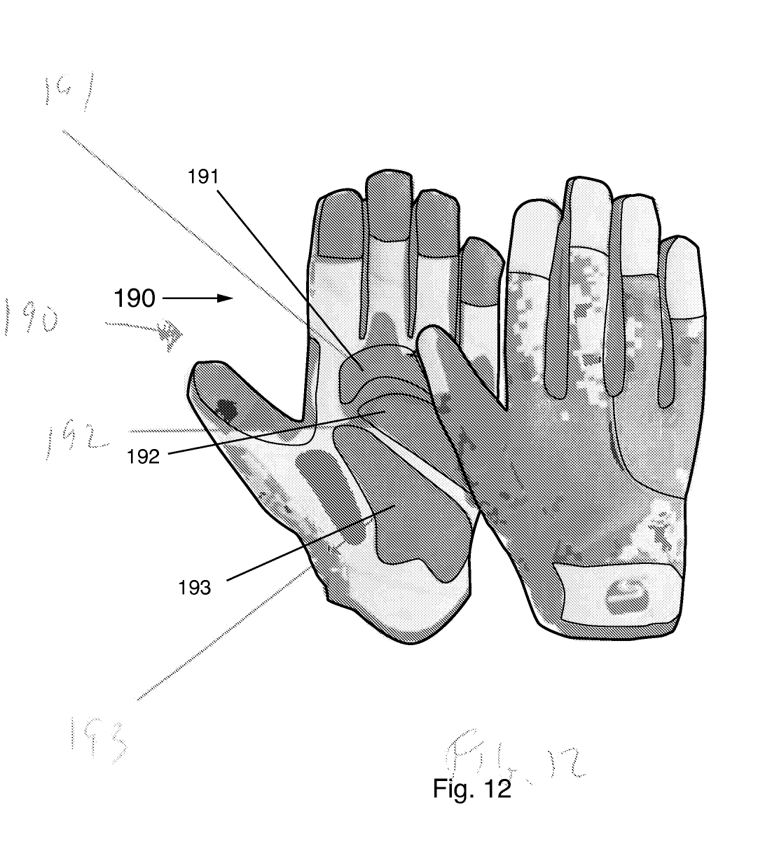

Reference is next made to FIG. 12 wherein gloves 190 constructed in accordance with another preferred embodiment of the invention are depicted. The gloves have similar methods of absorption enhancement of the beneficial materials which may be contacted in pads 191, 192 and 193, as well as the surface materials of glove 190. The absorption can be enhanced by abrasion, which assists with transdermal effects that benefit from heat. Similarly, sweat caused by the hand being maintained in the enclosed warm environment of the glove, creates sweat which aids with the osmotic treatment for absorbing the beneficial materials and pressure in portions of the glove where the wearer is gripping something aids in moving larger molecule sizes through the transdermal boarder. The beneficial contact surfaces of the glove can be both on the inner and outer surfaces of the glove because the wearer will come in contact with both surfaces during the course of their use of the gloves.

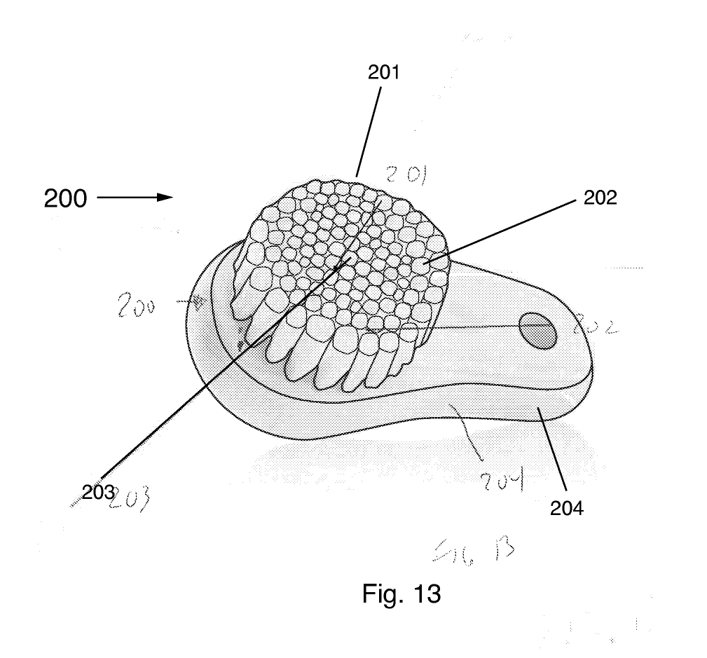

Reference is next made to FIG. 13 wherein a brush 200 constructed in accordance with another preferred embodiment of the invention is depicted. In the brush 200, handle 204 supports the brush portions including an abrasive ring 201 which can act to remove the barriers to entry of the beneficial materials and by heating up the surface of the user's skin in operation. Clay covered bristles 202 can act to absorb various toxins, drawing them out of and off of the user's skin. Finally, nano-mineral bristles 203 can be utilized to aid in depositing desirable minerals such as zinc and other zinc compounds, like zinc carbonate and zinc gluconate which are useful as nutrients, and as antibacterial and antimicrobial agents, magnesium oxide and other magnesium compounds and compositions and various other components as desirable vitamin materials created into nano-fiber form and other beneficial materials. Generally, zinc gluconate and magnesium oxide are preferably in the range of 50 nm to 50,000 nm in size, though it may also be larger. Other suitable compounds can be of similar or different dimensions.

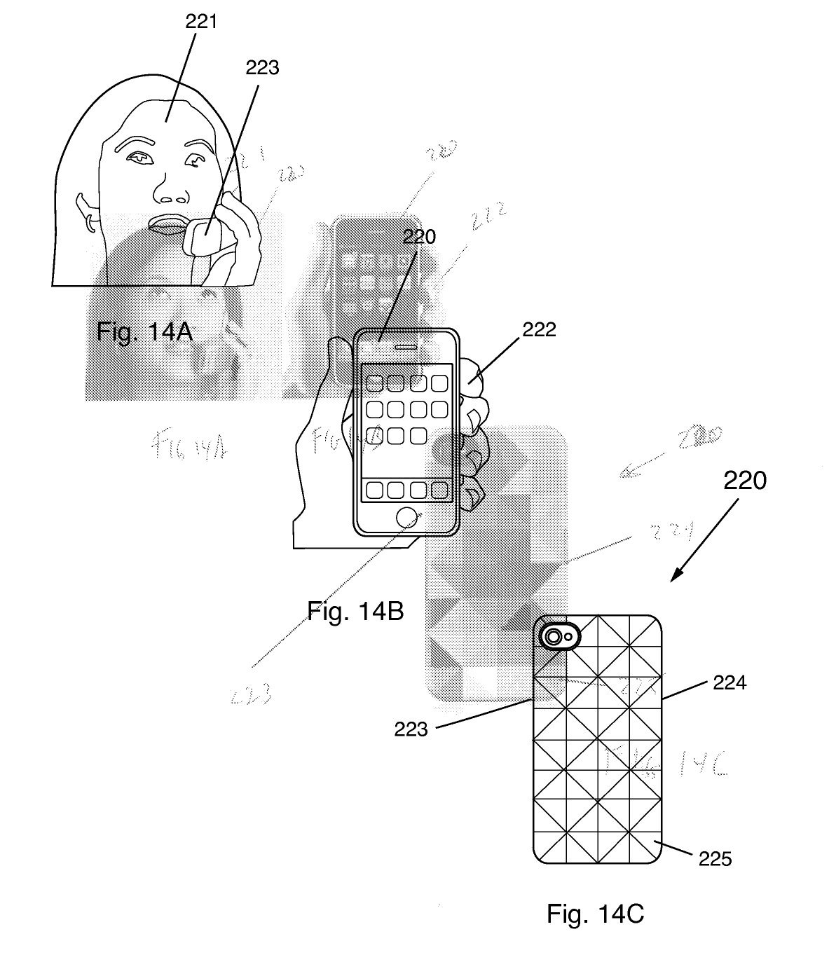

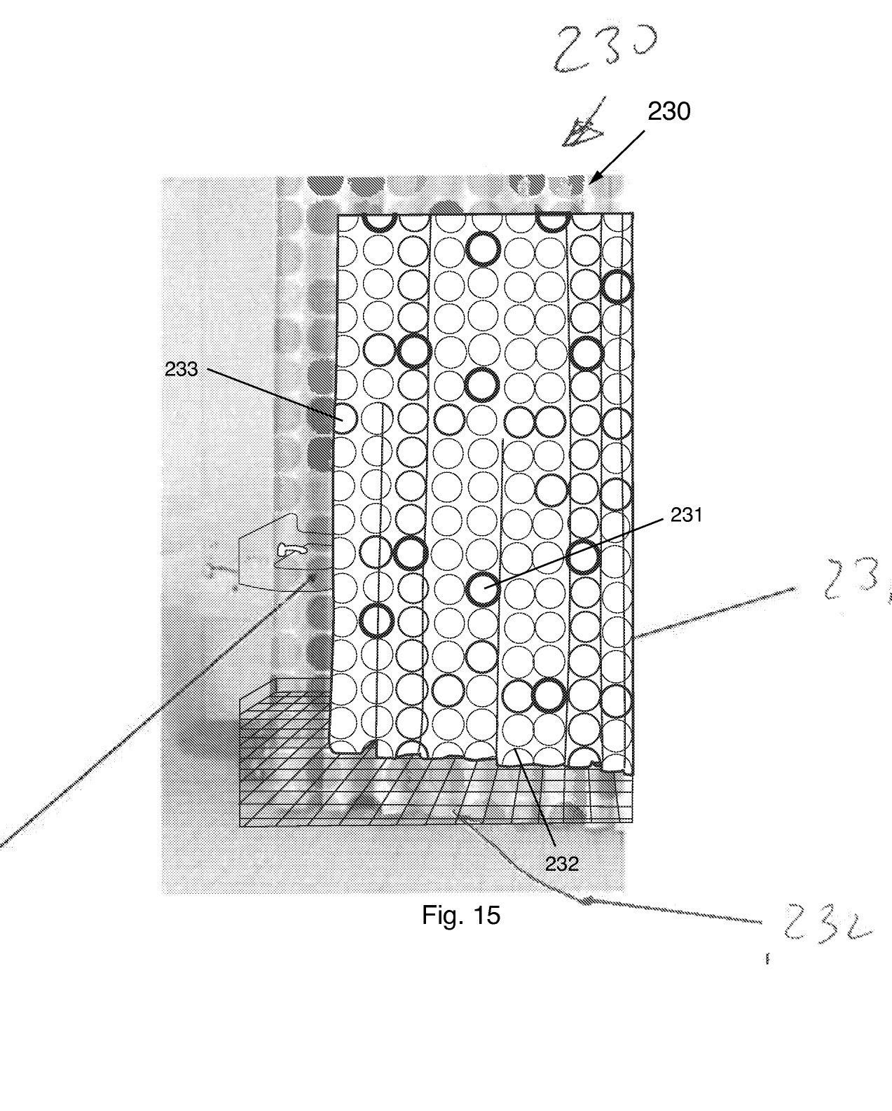

Reference is next made to FIGS. 14A, 14B and 14C wherein a cell phone 220 constructed in accordance with another preferred embodiment of the invention is depicted. As seen in FIG. 14A, the cell phone 220 is pressed up against the face of user 221. As conventionally known, the cell phone often rests up against the user's ear, cheek and lips. As shown in FIG. 14B, as the user manipulates cell phone 220 it is held in hand 222, including the fingers and palm of the user's hand 222. As shown in FIG. 14C, the back and sides of cell phone 200 can have nano-zinc zones 223, which cleanse the fingertips of germs, flecked ceramic zones 224, which detoxify the skin, and nano-magnesium zones 225, which increase whole-body health. The beneficial materials may be evenly distributed throughout the back and side surfaces of the cell phone, as well as surfaces on the front which are in contact with the user's body. The materials may be included by any of the approaches shown in FIGS. 3, 4, 5 and 6 to introduce the materials into the beneficial contact surface. The beneficial contact surfaces may be formed as part of the surface of the actual phone 220 or as a separate cell phone cover, as is commonly used for various fashion and design approaches currently.

Reference is next made to FIG. 13 wherein a shower curtain 230 constructed in accordance with a preferred embodiment is depicted. As shown, shower curtain 230 may have a variety of different surfaces which perform a variety of different features. For example, zone 231 may have a eucalyptus material added as a beneficial contact surface, which enhances respiratory health. An elemental zone 232 may have beneficial contact surfaces which, when contacted by the water within a shower environment, enhances the water treatment. Similarly, carbon zones 233 may be incorporated which absorb chlorine and provide a beneficial result to the shower user. Also, the surface of shower curtain 230 may be formed with either an application or integration of beneficial materials as shown and described in prior examples, which are relevant because shower users frequently come in contact with the inner surface with the shower curtain as they are bathing.

The wide variety of functional objects for which the beneficial contact surfaces may be used is only limited by the types of beneficial contact surface available and appropriate to the various delivery approach shown in FIGS. 3-7. In some applications multiple delivery approaches can be used. For example, a carpet might have the beneficial contact surface material integrated into the fibers of the pile. The base or backing of the carpet, to which the pile is attached could be enriched with additional beneficial contact surface material to refresh the beneficial contact surface material n the pile as people walk on the carpet. The bases' beneficial contact surface material could be delivered in different ways.

The density of the beneficial contact surface material can be varied depending on the rate of delivery desired and the expected bioabsorption rate. Also, the useful life of the article might impact the way the beneficial contact surface material is delivered. An object with a very long useful life like a doorknob would likely opt for more beneficial contact surface material and slower delivery than something like a cellphone cover which has an expected shorter lifespan before replacement or recycling which might be optimized for quicker delivery.

In current preferred embodiments of the invention both zinc and magnesium based compounds are utilized to create beneficial contact surfaces. Zinc is a metallic chemical element and is the first element of group 12 of the periodic table. Zinc is in some respects chemically similar to magnesium because its ion is of similar size and its only common oxidation state is +2. Zinc is fairly common in the Earth's crust and has five stable isotopes. The most common zinc ore is sphalerite (zinc blende), a zinc sulfide mineral. A variety of zinc compounds are commonly used, such as zinc carbonate and zinc gluconate (as dietary supplements), zinc chloride (in deodorants), zinc pyrithione (anti-dandruff shampoos), zinc sulfide (in luminescent paints), as zinc oxide, zinc methyl or zinc diethyl in the organic laboratory. Zinc ions are effective antimicrobial agents even at low concentrations. Zinc is an essential mineral of exceptional biologic and public health importance. Zinc deficiency affects about two billion people in the developing world and is associated with many diseases. In children, its absence or shortage causes growth retardation, delayed sexual maturation, infection susceptibility, and diarrhea, contributing to the death of about 800,000 children worldwide per year. Enzymes with a zinc atom in the reactive center are widespread in biochemistry, such as the alcohol dehydrogenase in humans. In humans, zinc plays ubiquitous biological roles. It interacts with a wide range of organic ligands and has roles in the metabolism of RNA and DNA, signal transduction, and gene expression.

In the US, the recommended dietary allowance of zinc is 8 mg/day for women and 11 mg/day for men. There are varying degrees of absorption by the body of zinc oxide, zinc sulfate, zinc gluconate and zinc picolinate or zinc citrate. Each of these may be utilized as the beneficial compound in the beneficial contact surface in accordance with the invention. There is a widespread deficiency of zinc around the world, particularly the developing world which has less access to good sources of nutrition. A beneficial contact surface in that environment might assist with the significant dietary deficiencies.

Magnesium is the fourth most abundant mineral in the body and is essential to good health. Fifty percent of total body magnesium is found in bone. The other half is bound predominantly inside cells of body tissues and cells. Only 1% of the magnesium is found in the blood, but the body works hard to maintain a constant level in the blood. Magnesium helps maintain normal muscle and nerve function, keeps heart rhythm steady, supports a healthy immune system and keeps bones strong. Magnesium also helps regulate blood sugar levels, promotes normal blood pressure and is known to be involved in energy metabolism and protein synthesis. Dietary magnesium is absorbed in the small intestines and excreted through the kidneys. With beneficial surface contact magnesium may be absorbed through the skin in small quantities. The RDA for magnesium in adult males is 400-420 mg/day and in adult females is 310-320 mg/day. Magnesium supplements usually combine magnesium with another substance such as a salt. Examples of magnesium supplements which may be appropriate as beneficial compounds for a beneficial contact surface include magnesium oxide, magnesium sulfate and magnesium carbonate.

The beneficial contact surfaces can be connected to the underlying contact surfaces of the articles in a variety of ways. In some situations the beneficial contact surface is simply glued, adhered or attached by traditional methods such as welding, gluing or mechanical means. In some situations the beneficial contact surface is formed by drying a liquid on the underlying contact surfaces, as in the drying of a paint or lacquer. Where the beneficial contact surface is formed as a skin for either a cellphone or laptop, the skin can be placed around the base article either through stretching like putting on a cellphone skin or by using a multipiece beneficial contact surface which snaps together or otherwise comes together. In others, like the example of the carpeting, the nanofibers or other small fibers of the beneficial contact surface are woven in with other fibers. In other case, where the beneficial contact surface compounds are added to the actual article the attachment is done in the manufacture of the article itself.

The beneficial surface contact compounds can be sourced in a variety of ways and then reduced in particle size to the appropriate size for the application indicated. The materials may be ground to the appropriate size by traditional means or may be formed as nanofibers through current nanotechnology means. The nanofabrication means are particularly useful with various of the delivery systems because the nanofiber forms of the beneficial contact surface compounds are more easily placed in a carrier, substrate, liquid or other fibers so that the beneficial contact surface compounds will come in contact with the user's skin as they use the articles and the bioabsorption process of absorbing the compound is enhanced.

Accordingly, various methods for providing beneficial contact surfaces for functional articles is provided. There are a number of different beneficial compounds described as well similar and additional compounds which can be utilized by the various processes describe and variations and combinations of those processes to functional articles and ornamental objects which have contact surfaces which come in contact with human beings or other non-human beings during their use.

It will thus be seen that the objects set forth above, among those made apparent in the preceding description, are efficiently obtained, and, since certain changes may be made in the above constructions without departing from the spirit and scope of the invention, it is intended that all matter contained in the above description or shown in the accompanying drawings shall be interpreted as illustrative, and not in a limiting sense.

It is also understood that the following claims are intended to cover all of the generic and specific features of the invention herein described and all statements of the scope of the invention, which, as a matter of language, might be said to fall there between.

What is claimed is:

1. An enhanced article, comprising:
a functional or ornamental article having at least one contact surface intended to come in contact with a human user;
beneficial contact surface means for covering the at least one contact surface and providing a beneficial surface contact compound adapted to come in contact with a human user touching the functional or ornamental article; and
connection means for securing the beneficial contact surface means to the article so that the at least one contact surface of the article is covered by the beneficial contact surface means;
whereby the presence of the beneficial surface contact compound in the beneficial contact surface means results in the exposure of the user's skin to the beneficial surface contact compound to allow absorption by the user.

2. The enhanced article of claim 1 wherein the beneficial contact surface means is a substrate embedded with the beneficial surface contact compound.

3. The enhanced article of claim 1 wherein the beneficial contact surface means is a liquid, gel or flowing solid embedded with the beneficial surface contact compound.

4. The enhanced article of claim 1 wherein the beneficial contact surface means is a woven, knit or nonwoven material where the beneficial surface contact compound is in fiber or nanofiber form and is integrated with the fibers of the woven, knit or nonwoven material.

5. The enhanced article of claim 4 wherein the article is a garment sock and the sock is knit from the beneficial surface contact compound or compounds integrated with the fibers used to make the sock.

6. The enhanced article of claim 4 wherein the article is a shoe and the shoe includes woven portions woven with the beneficial surface contact compound or compounds and traditional materials to form inside portions of the shoe which come in contact with the wearer's foot.

7. The enhanced article of claim 1 wherein the article is a package and beneficial surface contact compound is formed as part of the inks and dyes used to print on the package.

8. The enhanced article of claim 1 wherein the beneficial contact surface means is a solid member, embedded with the beneficial surface contact compound.

9. The enhanced article of claim 7 wherein the beneficial surface contact compound is a bioabsorbable material beneficial for health.

10. The enhanced article of claim 8 wherein the bioabsorbable material is magnesium oxide or zinc gluconate.

11. The enhanced article of claim 9 wherein the magnesium oxide or zinc gluconate is between about 50 nm and 50,000 nm.

12. The enhanced article of claim 1 wherein the beneficial surface contact compound includes zinc and/or magnesium.

13. The enhanced article of claim 1 wherein the beneficial surface contact compound includes a zinc compound.

14. The enhanced article of claim 1 wherein the beneficial surface contact compound includes a magnesium compound.

15. The enhanced article of claim 1 wherein the beneficial surface contact compound includes a nanofiber form of a magnesium compound.

16. The enhanced article of claim 1 wherein the beneficial surface contact compound includes a nanofiber form of a zinc compound.

17. The enhanced article of claim 1 wherein the beneficial surface contact compound is formulated so as to be bioabsorbable by the user's skin upon contact.

18. The enhanced article of claim 1 wherein the article is a door knob and the beneficial surface contact compound is embedded in a substrate covering the door knob handled by the user in operating the door knob.

19. The enhanced article of claim 1 wherein the article is a cellphone and the beneficial surface contact compound is embedded in a cellphone cover which covers at least the regions of the cellphone which are handled and pressed against the user's face or hands when in use.

20. A process for providing a beneficial contact surface for articles which come in contact with a user's skin, comprising:
identifying the surfaces of an article which come in contact with a user's skin;
selecting a beneficial surface contact compound which provides the desired beneficial contact benefits for users of the article;
embedding the beneficial surface contact compound in an appropriate carrier;
securing the carrier to the article so that the carrier, with the embedded beneficial surface contact compound, acts as the surface contacts of the article as it comes in contact with a user's skin;
whereby the beneficial surface contact compound can be bioabsorbed by the user when in contact with the carrier.

21. The process of claim 20 for providing a beneficial contact surface for articles which come in contact with a user's skin, wherein the selecting of the beneficial surface contact compound is accomplished by matching the desired beneficial characteristics of the contact with a desired compound.

22. The process of claim 21 wherein the beneficial contact surface compound is selected from the group of zinc and magnesium compounds.

23. The process of claim 20 wherein the beneficial contact surface compound is embedded in the carrier by reducing the size of each piece of the beneficial contact surface compound to an appropriate size and then mixing the beneficial contact surface compound in a liquid form of the carrier.

24. The process of claim 20 wherein the beneficial contact surface compound is embedded in the carrier by reducing the size of each piece of the beneficial contact surface compound to an appropriate size and then embedding the beneficial contact surface compound in a solid form of the carrier.

25. The process of claim 20 wherein the selecting of a beneficial contact surface compound is performed by choosing from a zinc or magnesium compound.
